(19) United States
(12) Patent Application Publication
(10) Pub. No.: US 2022/0233164 A1
(43) Pub. Date: Jul. 28, 2022

(54) Ultrasound Probe

(71) Applicant: BK Medical ApS, Herlev (DK)

(72) Inventors: Jacob Bjerring Olesen, Copenhagen (DK); Carlos Armando Villagomez Hoyos, Copenhagen (DK); Rasmus Holdensgaard Rossen, Vanlose (DK); Fredrik Gran, Limhamn (SE)

(73) Assignee: BK Medical ApS, Herlev (DK)

(21) Appl. No.: 17/616,332

(22) PCT Filed: Jun. 3, 2019
(86) PCT No.: PCT/IB2019/054583

§ 371 Date: Dec. 3, 2021

(51) Int. Cl. A61B 8/12; A61B 8/00; A61B 8/08

(52) U.S. Cl. CPC A61B 8/12; A61B 8/4488; A61B 8/4254; A61B 8/483; A61B 8/4472; A61B 8/445

(57) ABSTRACT

A system (102) includes an ultrasound probe (104). The ultrasound probe includes a probe head (202), a handle (208), and an elongate shaft (226) disposed between and coupling the probe head and the handle. The probe head houses a transducer array (114). The elongate shaft includes a first portion (232) coupled to the probe head and a second portion (234) coupled to the handle. The second portion includes a first end region (238) coupled to the handle. The second portion further includes a second end region (236) extending above the handle and coupled to the first portion such that a line of site from behind the probe to the probe head is visually unobstructed by the handle.

### TECHNICAL FIELD

The following generally relates to ultrasound imaging and more particularly to an ultrasound probe, and is described with particular application to neurosurgery, and is amenable to other applications in which a probe head with a transducer is inserted into a natural or artificial cavity in a subject (e.g., human or animal) or an object (e.g., non-human/animal) to image a surface of the natural or artificial cavity.

### BACKGROUND

Ultrasound imaging has provided useful information about the interior characteristics of an object or subject under examination. For example, intraoperative ultrasound imaging has been used in brain tumor surgery for detecting and localizing tumor remnants Unlike other imaging modalities, e.g. magnetic resonance imaging (MRI) and computed tomography (CT) in neurosurgery, ultrasound offers a real-time display of anatomy and/or function during a procedure. In one instance, the real-time display allows for determining a relevancy of a pre-operative navigation image. For instances, when a brain shift occurs the pre-operative image used by the navigation/tracking system may become obsolete and unusable to the surgeon for the procedure. In another example, the real-time display allows to detect a presence of blood, assess whether a vessel needs to be avoided or clipped, identify a functional area to avoid, etc.

However, ultrasound transducers in neurosurgery are designed for scanning from the top of the cavity, and not designed for reaching the bottom of the resection cavity, e.g., due to their bulk sizes. That is, the ultrasound probes do not carry a form factor that allows easy scanning onto the bottom of the resection cavity and keeps the surgeon line of sight. As a consequence, saline is added into the resection cavity for enabling acoustic coupling between the bottom of the resection zone and the transducer surface. Unfortunately, the introduction of saline into the procedure interrupts workflow as saline has to be added and then subsequently drained. In addition, the saline manifests as image brightness artifact, e.g., introduced from overcompensating the received signal due to a mismatch in attenuation coefficients between saline and brain tissue.

### SUMMARY

Aspects of the application address the above matters, and others.

In one aspect, a system includes an ultrasound probe. The ultrasound probe includes a probe head, a handle, and an elongate shaft disposed between and coupling the probe head and the handle. The probe head houses a transducer array. The elongate shaft includes a first portion coupled to the probe head and a second portion coupled to the handle. The second portion includes a first end region coupled to the handle. The second portion further includes a second end region extending above the handle and coupled to the first portion such that a line of site from behind the probe to the probe head is visually unobstructed by the handle.

In another aspect, a method includes positioning a head of an ultrasound probe in a cavity within an object. The ultrasound probe includes a transducer array in the head, a handle, and an elongate shaft between the head and the handle. The elongate shaft includes a linear portion coupled to the head and a non-linear portion coupled to the handle. The non-linear portion protrudes above the handle and is coupled to the first portion such that a line of site from behind the probe to the head is visually unobstructed by the handle. The method further includes transmitting ultrasound signals with the transducer array. The method further includes receiving echo signals with the transducer array. The method further includes generating an image of an inside of the cavity based on the received echo signals.

In yet another aspect, an ultrasound system includes a console and an ultrasound probe in electrical communication with the console. The ultrasound probe includes a probe head that houses a transducer array, a handle; and an elongate shaft coupling the probe head and the handle. The elongate shaft positions the probe head above the handle so that a line of site from behind the probe to the probe head is visually unobstructed by the handle.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings, in which like references indicate similar elements and in which:

FIG. 1 schematically illustrates an example ultrasound imaging system including a console and a probe, in accordance with an embodiment(s) herein;

FIG. 2 schematically illustrates an example of the probe, in accordance with an embodiment(s) herein;

FIG. 3 schematically illustrates an example of a rectangular shaped face, in accordance with an embodiment(s) herein;

FIG. 4 schematically illustrates another example of a rounded rectangular shaped face, in accordance with an embodiment(s) herein;

FIG. 5 schematically illustrates an example of a square shaped face, in accordance with an embodiment(s) herein;

FIG. 6 schematically illustrates another example of a rounded square shaped face, in accordance with an embodiment(s) herein;

FIG. 7 schematically illustrates an example of a circular shaped face, in accordance with an embodiment(s) herein;

FIG. 8 schematically illustrates another example of an elliptical square shaped face, in accordance with an embodiment(s) herein;

FIG. 9 schematically illustrates another example of the probe, in accordance with an embodiment(s) herein;

FIG. 10 schematically illustrates yet another example of the probe, in accordance with an embodiment(s) herein;

FIG. 11 schematically illustrates still another example of the probe, in accordance with an embodiment(s) herein;

FIG. 12 schematically illustrates another example of the ultrasound imaging system further including a tracking system, in accordance with an embodiment(s) herein;

FIG. 13 schematically illustrates an example method, in accordance with an embodiment(s) herein;

FIG. 14 schematically illustrates an example configuration for 3-D and/or 4-D imaging, in accordance with an embodiment(s) herein;

FIG. 15 schematically illustrates another example configuration for 3-D and/or 4-D imaging, in accordance with an embodiment(s) herein; and

FIG. 16 schematically illustrates still another example configuration for 3-D and/or 4-D imaging, in accordance with an embodiment(s) herein.

### DETAILED DESCRIPTION

FIG. 1 illustrates an example imaging system 102 such as an ultrasound imaging system/scanner. The imaging system 102 includes a probe 104 and a console 106, which interface with each other through suitable complementary wireless interfaces 108 and 110 and/or hardware (e.g., cable connectors and a cable, etc.). With a wireless probe, the probe 104 can be placed on an instrument table next to an examination region, which can improve workflow efficiency and/or allow acquired data to be wirelessly transmitted to a computing device for processing.

The probe 104 includes a transducer array 114 with one or more transducer elements 116. The one or more transducer elements 116 includes a capacitive micromachined ultrasonic transducer (cMUT), thick film print, piezoelectric-composite and/or other type of transducer material. In one instance, the transducer material provides optimal near-field image quality. In another instance, the transducer material is based on other criteria. The one or more transducer elements 116 are configured to convert an excitation electrical pulse into an ultrasound pressure field and convert a received ultrasound pressure field (an echo) into electrical (e.g., a radio frequency (RF)) signal.

The one or more transducer elements 116 is arranged as a 1-D or 2-D, linear, curved and/or otherwise shaped, fully populated or sparse, etc. array. In one instance, a pitch is selected to ensure a wide steering angle in either 2-D or 3-D and/or a footprint of the array 114 is set to fit a smallest cavity ducted by the user. For example, in one instance the pitch is half a wavelength. The one or more transducer elements 116 are configured to transmit in a range of one (1) to fifty (50) megahertz (MHz). For example, for neurosurgery application, in one instance, the one or more transducer elements 116 are configured to transmit at a frequency that is in a range of five (5) to eighteen (18) MHz.

In one instance, the probe 104 is configured for one-dimensional (1-D) imaging. Additionally, or alternatively, the probe 104 is configured for two-dimensional (2-D) imaging Additionally, or alternatively, the probe 104 is configured for three-dimensional (3-D) imaging. Additionally, or alternatively, the probe 104 is configured for four-dimensional (4-D) imaging. In one instance, for 3-D and/or 4-D imaging, the transducer array is swept to acquire volumetric data using mechanical and/or electronical approaches. Mechanical approaches include tilting the transducer via a motor inside the probe and/or otherwise, and electronical approaches include electronically steering the emitted ultrasound beam.

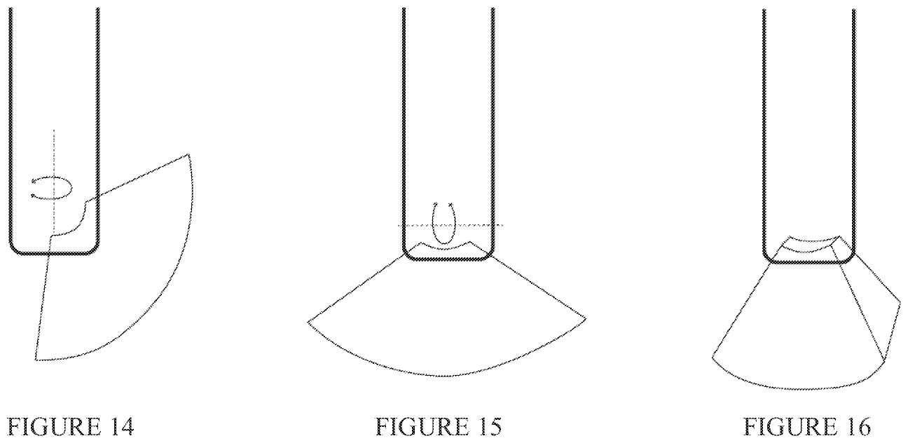

FIGS. 14, 15 and 16 schematically illustrate different configurations of the one or more transducer elements 116 for 3-D and/or 4-D imaging. It is to be understood that other configurations are also contemplated herein. FIG. 14 schematically illustrates a configuration in which the transducer array 114 produces an image plane 1402 that rotates around a long axis 1404 of an elongate end 1406 of the probe 104. In this embodiment, an axis of rotation 1408 is the long axis 1404. In the FIG. 15 schematically illustrate a configuration in which the transducer array 114 rotates an image plane 1502 through a position where the image plane 1502 extends out axially from the long axis 1404 (shown) to other positions, such a where the image plane 1502 is rotated about a short axis 1504 to extend transverse to the long axis 1404. In this embodiment, an axis of rotation 1506 is the short axis 1504. FIG. 16 schematically illustrate a configuration in which the transducer array 114 transmits a 3-D beam 1602 that extends out from the long axis 1404 from a front 1604 of the elongate end 1406.

Returning to FIG. 1, as described in greater detail below, in one instance the probe 104 is structurally configured for access into a (natural or artificial) cavity, including reaching a bottom of the cavity for scanning an interior of the cavity, and structurally configured to provide an unobstructed line of sight by the use to a tip of the probe 104 (e.g., the probe head). As such, the probe 104 mitigates having to add saline or other material into the cavity for enabling acoustic coupling between probe and the medium of interest. In one instance, this results in a clear ultrasound image without contamination of brightness/enhancement artifact and without interrupting the user's workflow.

The console 106 includes transmit circuitry (TX) 118 configured to generate the excitation electrical pulses and receive circuitry (RX) 120 configured to process the RF signals, e.g., amplify, digitize, and/or otherwise process the RF signals. The console 106 further includes a switch (SW) 122 configured to switch between the TX 118 and the RX 120 for transmit and receive operations, e.g., by electrically connecting and electrically disconnecting the TX 118 and the RX 120.

The console 106 includes further an echo processor 124 configured to process the signal from the RX 120. For example, in one instance the echo processor 124 is configured to beamform (e.g., delay-and-sum) the signal to construct a scanplane of scanlines of data. The echo processor 124 can be implemented by a hardware processor such as a central processing unit (CPU), a graphics processing unit (GPU), a microprocessor, etc.

The console 106 further includes a display 126. The output of the echo processor 124 is scan converted to the coordinate system of the display 126 and displayed as images via the display 126. In one instance, the scan converting includes changing the vertical and/or horizontal scan frequency of signal based on the display 126. The scan converter can be configured to employ analog scan converting techniques and/or digital scan converting techniques. In a variation, the display 126 is separate from the console 106 and electrically connected thereto.

The console 106 further includes a user interface 128, which includes one or more input devices (e.g., a button, a touch pad, a touch screen, etc.) and one or more output devices (e.g., a display screen, a speaker, etc.). The user interface 128, in one instance, allows a user to manipulate a displayed image, e.g., zoom, pan, rotate, set/change a gain value, change a display mode, etc.

The console 106 further includes a controller 130 configured to control one or more of the probe 104, the transmit circuitry 118, the receive circuitry 120, the switch 122, the echo processor 124, the display 126, the user interface 128, and/or one or more other components of the imaging system 102. The controller 130 can be implemented by a hardware processor such as a CPU, a GPU, a microprocessor, etc.

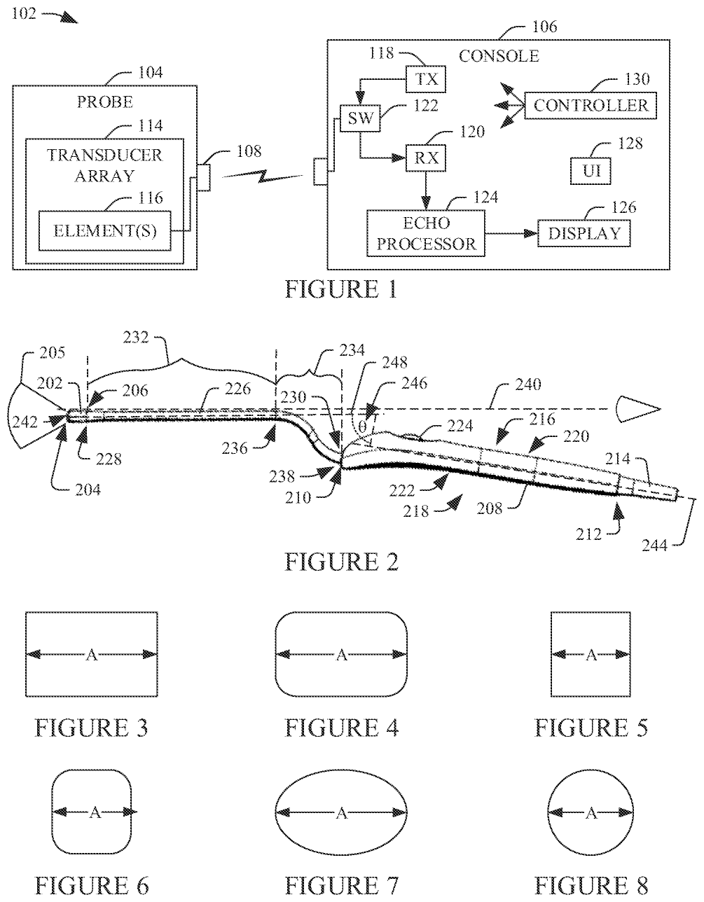

FIG. 2 illustrates a non-limiting example of the probe 104.

In this example, the probe 104 includes a probe head 202 with a first end region 204 and a second end region 206, which spatially opposes the first end region 204. The transducer array 114 (not visible) is disposed and housed within the probe head 202. In this example, the probe 104 is configured as an end fire probe, with a transducing side of the transducer array 114 facing out of the first end region 204 of the probe head 202 such that a transmitted beam 205 traverses a direction out of the first end region 204.

The probe 104 further includes a handle 208 with a first end region 210 and a second end region 212, which spatially opposes the first end region 210. Electronics 214 for routing electrical signals indicative of received ultrasound pressure fields from the probe 104 to a device remote from the probe 104 are disposed at the second end region 212. As briefly discussed above, electronics 214 may include a cable and/or a wireless interface.

The illustrated handle 208 is cylindrical in shaped and includes a top 216 and a bottom 218 coupled together by sides 220 and 222. The illustrated handle 208 further includes a least one physical control 224 (e.g., a button) located on the top 216. In one instance, the control 224 is configured to activate and deactivate transmission and reception. In another embodiment, the control 224 is otherwise located (e.g., on the bottom 218, etc.) and/or the handle 208 includes another type and/or control(s). For example, in another instance, the handle 208 includes a freeze control button.

The probe 104 further includes an elongated shaft 226 with a first end region 228 and a second end region 230, which spatially opposes the first end region 228. The second end region 206 of the probe head 202 is coupled to the first end region 228 of the shaft 226, and the first end region 210 of the handle 208 is coupled to the second end region 228 of the shaft 226. In this example, the shaft 226 includes a first portion 232 and a second portion 234. In this example, the first portion 232 is generally linear shaped, and the second portion 234 is non-linear shaped. In this embodiment, a length of the shaft 226 is between three (3) and ten (10) millimeters (mm), such as five (5) mm, six and a half (6.5) mm, etc.

In this example, the second portion 234 generally is sigmoid or “S” shaped with a first end region 236 and a second end region 238 and rigid/non-flexible. In other embodiments, the second portion 234 is otherwise shaped (e.g., linear) and/or flexible/non-rigid. The second end region 238 is coupled to the handle 208 between the top 216 and the bottom 218, which causes the first end region 236 to be raised above the handle 208, providing a direct line of site 240 to the probe head 202 by a user of the probe 104, unobstructed from the handle 208. In one instance, this aids the user with navigating the probe head 202 into a cavity for imaging a surface of an inside of the cavity proximate with the surface without having to add a coupling fluid, such as saline or the like, to the cavity.

In one instance, the shape of the shaft 226 mimics that shape of a tool utilized during a resection or other procedure. For example, in one instance the shape of the shaft 226 imitates surgical instruments that surgeons are already using throughout a procedure. That is, the probe 104 has a bend between the probe head 202 that enters the cavity and the handle 208 on which a surgeon operates it. In this example, the bend in part of the second portion 234 and has an angle for the uninterrupted line of sight 240 from the eyes of the operator to the bottom of the cavity.

A face 242 of the first end region 204 of the probe head 202 can be variously shaped. FIGS. 3-8 schematically illustrate several examples. FIG. 3 depicts a rectangular shaped face 242. FIG. 4 depicts a rectangular shaped face 242 with rounded corners. FIG. 5 depicts a square shaped face 242. FIG. 6 depicts a square shaped face 242 with rounded corners. FIG. 7 depicts an ellipse shaped face 242. FIG. 8 depicts a circular shaped face 242. In one instance, a longest side “A” or diameter of the face 242 is between five (5) and twenty (20) millimeters (mm), such as fourteen (14) mm or other dimension such that it can fit into a burr hole cavity.

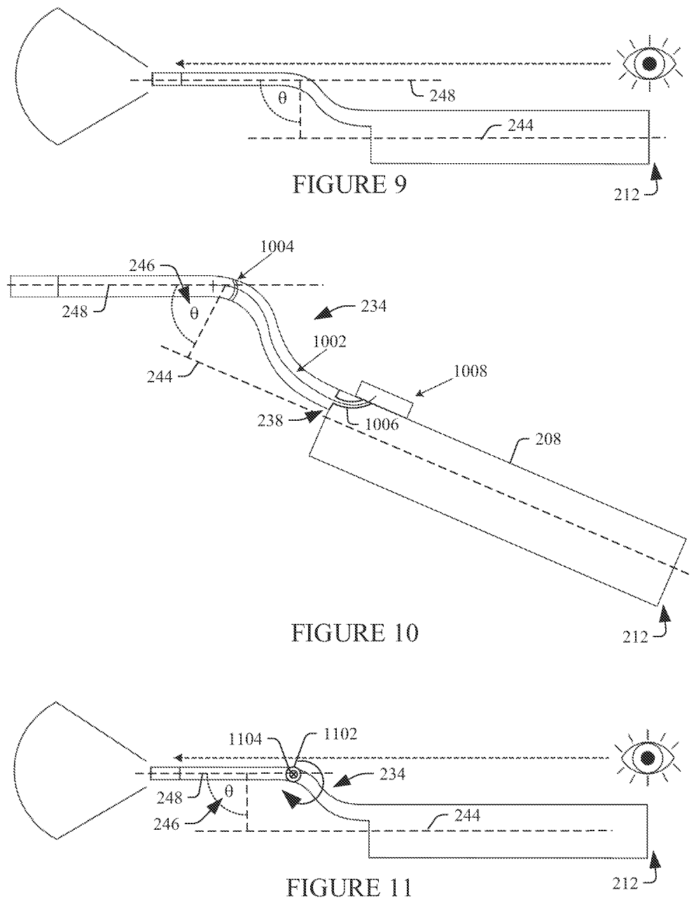

In FIG. 2, a long axis 244 of the handle 208 is at a fixed angle (θ) 246 between zero (0) and ninety (90) degrees with respect to a long axis 248 of the first portion 232 of the shaft 226. In another instance, as shown in FIG. 9, the angle (θ) 246 is fixed at ninety (90) degrees, and the long axis 244 of the handle 208 and the long axis 248 of the shaft 226 are parallel. In another embodiment, the angle (θ) 246 is between that shown in FIGS. 2 and 9. In yet another instance, the angle (θ) 246 is fixed at an angle less than that shown in FIG. 2, including a negative angle where the second end region 212 of the probe 104 faces the direction of the beam 205.

In another instance, the angle (θ) 246 is adjustable. FIG. 10 shows an embodiment in which the “S” shaped portion 234 is configured to flex and includes a tension wire 1002, which is statically connected at a pivot joint 1004, free at the first end region 238, and fed through a hollow channel 1006 in the handle 208. Pulling the tension wire 1002 out of the hollow channel 1006 causes the “S” shaped portion 234 to flex inward/downward, decreasing the angle (θ) 246. A fastener 1008, such as a clamp or the like, when engaged, holds the tension wire 1002 at a current position. Disengaging the fastener 1008 allows the tension wire 1002 to move to increase or decrease the angle (θ) 246.

In one instance, the “S” shaped portion 234 is configured to flex between an angle of θ=zero (0) degrees (where the axis 244 is perpendicular to the axis 248) and θ=ninety (90) degrees (where the axis 244 is parallel to the axis 248). In yet another embodiment, the “S” shaped portion 234 is configured to flex less than ninety (90) degrees. In still another embodiment, the “S” shaped portion 234 is configured to flex more than ninety (90) degrees. For example, in this embodiment the “S” shaped portion 234 flexes such that the angle (θ) 246 is negative and the second end region 212 of the probe 104 flexes and faces the direction of the beam 205.

FIG. 11 shows another embodiment in which the “S” shaped portion 234 is connected to the shaft 226 via a pivot joint 1102 that includes a fastener 1104, such as a set screw, or the like, that when engaged holds the “S” shaped portion 234 at a current position relative to the shaft 226. Disengaging the fastener 1104 allows the “S” shaped portion 234 to pivot. Similar to FIG. 10, the “S” shaped portion 234 can be configured to pivot less than ninety (90) degrees, ninety (90) degrees or more than ninety (90) degrees, including from where the axes 244 and 248 are parallel to where the second end region 212 of the probe 104 faces the direction of the beam 205, which is opposite to the direction illustrated in FIG. 11.

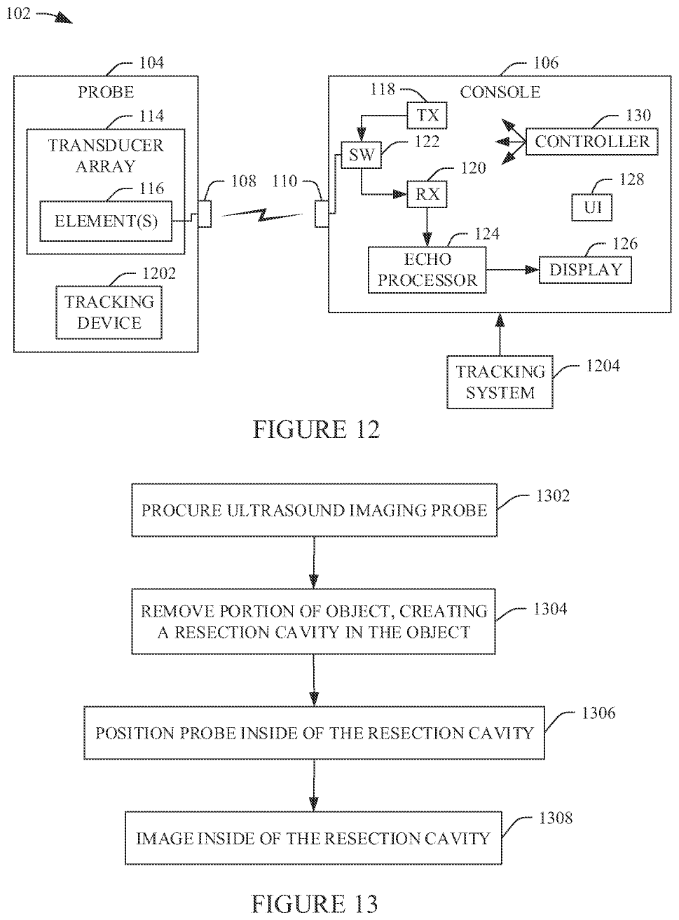

FIG. 12 illustrates another non-limiting example of the system 102.

In this variation, the probe 104 further includes an internal and/or external tracking device(s) 1202 and the console 106 further includes a probe tracking system 1204, which tracks a spatial orientation of the probe 104 based on a signal from the tracking device(s) 1202. Suitable tracking devices include electromagnetic, optical, etc.

With an electromagnetic tracking device, in one instance tracking coils are included in the handle 208, the shaft 226, and/or the head 202. The tracking system 1204 measures a magnetic field strength of the coils, which depends on a distance and direction of the coils to the tracking system 1204, and the strength and direction is used to determine location and orientation of the probe 104.

With an optical tracking device, in one instance a fiducial target is placed on the handle 208, e.g., adjacent the first end region 210 of the handle 208, which corresponds to a location between the “S” shaped portion 234 of the elongate shaft 226 and a user's hand on the handle 208. In one instance, this ensures optimal line of sight between operator and cavity zone and between an optical tracking system and the handle 208. The tracking system 1204 includes a video camera or the like that records the spatial orientation of the fiducial to determine location and orientation of the probe 104.

Suitable tracking is discussed in Birkfellner et al., “Tracking Devices,” In: Peters T., Cleary K. (eds) Image-Guided Interventions. Springer, Boston, Mass., 2008, and U.S. patent application US 2010/0298712 A1, filed Feb. 10, 2010, and entitled “Ultrasound Systems Incorporating Position Sensors and Associated Method,” which is incorporated herein by reference in its entirety. Other approaches are also contemplated herein.

FIG. 13 illustrates an example method in accordance with an embodiment herein.

It is to be appreciated that the order of the below acts is not limiting, and in other embodiments, there may be more, less and/or different acts.

At 1302, the ultrasound imaging probe 104 is procured.

At 1304, a portion of an object within the object is removed, creating a cavity.

At 1306, the head 202 of the ultrasound imaging probe 104 is positioned in the cavity.

At 1308, the ultrasound imaging probe 104 is activated to image an inside of the cavity.

The application has been described with reference to various embodiments. Modifications and alterations will occur to others upon reading the application. It is intended that the invention be construed as including all such modifications and alterations, including insofar as they come within the scope of the appended claims and the equivalents thereof.

1. A system, comprising:
an ultrasound probe, including:
a probe head housing a transducer array;
a handle; and
an elongate shaft disposed between and coupling the probe head and the handle, the elongate shaft including:
a first portion coupled to the probe head; and
a second portion coupled to the handle, the second portion, including:
a first end region coupled to the handle; and
a second end region extending above the handle and coupled to the first portion such that a line of site from behind the probe to the probe head is visually unobstructed by the handle.

2. The system of claim 1, wherein the second portion is sigmoid shaped or “S” shaped.

3. The system of claim 1, wherein the second portion is non-linear.

4. The system of claim 1, wherein the second portion is linear.

5. The system of claim 1, wherein the second portion of the elongate shaft is statically coupled to the first portion of the shaft.

6. The system of claim 1, wherein the second portion is rigid.

7. The system of claim 1, wherein the second portion is configured to flex relative to the first portion of the shaft.

8. The system of claim 7, wherein the second portion is configured to flex between one position in which a long axis of the handle is at a first angle with respect to a long axis of the first portion of the elongate shaft and another position in which the long axis of the handle is at a second different angle with respect to the long axis of the first portion of the elongate shaft.

9. The system of claim 1, further comprising:
a pivot joint that couples the first and second portions of the elongate shaft.

10. The system of claim 9, wherein the pivot joint is configured to pivot between one position in which a long axis of the handle is at a first angle with respect to a long axis of the first portion of the elongate shaft and another position in which the long axis of the handle is at a second different angle with respect to the long axis of the first portion of the elongate shaft.

11. The system of claim 1, wherein the transducer array is configured to mechanically rotate to move a transmitted ultrasound beam at least one of around the probe head or about the probed head for 3-D and/or 4-D imaging.

12. The system of claim 1, wherein the transducer array is configured to transmit a 3-D beam for 3-D and/or 4-D imaging.

13. The system of claim 1, further comprising:
a wireless interface configured to communicate with a complementary wireless interface of an ultrasound console.

14. The system of claim 1, further comprising:
a tracking device for tracking a spatial location and orientation of the probe, wherein the tracking device is selected from a group consisting of: an electromagnetic sensor or an optical fiduciary.

15. A method, comprising:
positioning a head of an ultrasound probe in a cavity within an object,
wherein the ultrasound probe includes a transducer array in the head, a handle, and an elongate shaft between the head and the handle,
wherein the elongate shaft includes a linear portion coupled to the head and a non-linear portion coupled to the handle, and
wherein the non-linear portion protrudes above the handle and is coupled to the first portion such that a line of site from behind the probe to the head is visually unobstructed by the handle; and
transmitting ultrasound signals with the transducer array;
receiving echo signals with the transducer array; and
generating an image of an inside of the cavity based on the received echo signals.

16. The method of claim 15, further comprising:
imaging the inside of the cavity is image without adding a coupling material to the cavity; and
generating the image without coupling material brightness artifact.

17. The method of claim 15, further comprising:
rotating the transducer array for at least one of 3-D or 4-D imaging.

18. The method of claim 15, further comprising:
tracking a spatial orientation of the probe with respect to the cavity.

19. The method of claim 15, further comprising:
adjusting a spatial position of the handle with respect to the head via a portion of the elongate shaft while maintaining the visually unobstructed line of site from behind the probe to the head.

20. An ultrasound system, comprising:
a console;
an ultrasound probe in electrical communication with the console, wherein the ultrasound probe includes:
a probe head that houses a transducer array;
a handle; and
an elongate shaft coupling the probe head and the handle,
wherein the elongate shaft positions the probe head above the handle so that a line of site from behind the probe to the probe head is visually unobstructed by the handle.
